(19) United States
(12) Patent Application Publication
(10) Pub. No.: US 2009/0280162 A1
(43) Pub. Date: Nov. 12, 2009

(54) LAYERED WOUND DRESSING

(76) Inventors: Jürgen Wegmann, Stockach (DE); Erich Odermatt, Schaffhausen (CH); Bernd Blender, Hohentengen (DE)

Correspondence Address: IP GROUP OF DLA PIPER LLP (US), ONE LIBERTY PLACE, 1650 MARKET ST, SUITE 4900, PHILADELPHIA, PA, 19103, US

(21) Appl. No.: 12/297,657

(22) PCT Filed: Apr. 19, 2007
(86) PCT No.: PCT/EP2007/003414

§ 371 Date: Dec. 2, 2008

(30) Foreign Application Priority Data
Apr. 20, 2006 (DE) 10 2006 020 498.0

(51) Int. Cl. A61K 9/00; B32B 37/02; A61P 17/02

(52) U.S. Cl. 424/447; 156/60

(57) ABSTRACT

A wound dressing including at least one biocompatible support material and at least one polysaccharide as a hemostatic means (hemostyptic) and/or that unites severed regions of body tissue, and having a layered structure of at least one layer of the support material and at least one layer of the polysaccharide.

### RELATED APPLICATIONS

This is a §371 of International Application No. PCT/EP2007/003414, with an international filing date of Apr. 19, 2007 (WO 2007/121912 A2, published Nov. 1, 2007), which is based on German Patent Application No. 10 2006 020 498.0, filed Apr. 20, 2006.

### TECHNICAL FIELD

This disclosure relates to wound dressings having at least one biocompatible support material and at least one polysaccharide, to methods for producing them and to the use of the wound dressings.

### BACKGROUND

The treatment of wounds, especially the uniting of severed regions of body tissue (incisions) is central to modern health care. Diverse wound dressings are employed advantageously for this purpose. Thus, for example, collagen fabrics are routinely employed for the hemostatic management of internal wounds. For example, a fabric-like wound dressing of this type is marketed commercially by the assignee under the name Lyostypt®.

For successful wound management, in most cases it is particularly necessary for the wound dressing material to have hemostatic properties and for the wound dressing itself to adhere adequately to the wound area to be treated. In many cases it is necessary to provide the wound dressings with adhesive compositions to avoid slippage or detachment of the wound dressings on the wound area to be managed. The adhesive compositions are frequently compositions based on proteins, in particular proteins of the coagulation cascade, for example thrombin or fibrinogen.

There has recently been increasing development also of wound dressings based on polysaccharide derivatives. These derivatives are in particular oxidized polysaccharides having in particular carboxyl groups. Thus, for example, wound dressings based on cellulose having carboxyl groups are routinely employed in modern surgical care. However, a disadvantage in this connection is in principle the denaturing effect of polysaccharides oxidized in this way on possible protein constituents of the wound dressing.

A further group of wound dressings is based on polysaccharides having aldehyde groups (polyaldehydic). These wound dressings have the advantage of a distinctly improved adhesion to the region of body tissue to be treated. The improved adhesive properties, compared with other wound dressings, are based in particular on the reactivity of the aldehyde groups of the oxidized polysaccharides vis-à-vis the amino groups of the body tissue proteins, bringing about successful adhesion of the wound dressings to the particular region of body tissue. A wound dressing of this type is disclosed, for example, in WO 2004/091677 A1. Further examples of such wound dressings may be found in EP 1 430 911 A2, EP 1 378 255 A2 and EP 1 424 085 A1.

However, a problem is that wound dressings based on polysaccharide derivatives frequently have only moderate, or in some cases even poor, biocompatibility. This may cause in particular undesired traumatizations in the region of the wound area to be managed.

It could therefore be helpful to provide a wound dressing which avoids the above problems and to have high biocompatibility and display good adhesion to the wound area to be managed.

### SUMMARY

We provide a wound dressing including at least one biocompatible support material and at least one polysaccharide as a hemostatic means (hemostyptic) and/or that unites severed regions of body tissue, and having a layered structure of at least one layer of the support material and at least one layer of the polysaccharide.

We also provide a process for producing a wound dressing including at least one biocompatible support material and at least one polysaccharide including providing at least one dispersion of the support material in a liquid dispersion medium, cooling and solidifying the dispersion to form at least one solid layer of the support material, applying the polysaccharide to the solid support layer, cooling and solidifying the applied polysaccharide to form at least one solid layer of the polysaccharide on the support layer, and removing the dispersion medium to unite the layers together.

### BRIEF DESCRIPTION OF THE DRAWINGS

Further details and features of our wound dressings and processes are evident from the following description of preferred constructions in combination with the figures and examples. Individual features can be implemented alone or in combination with other features. All the figures are herby made contents of this description by express reference.

The figures show the following:

FIG. 1: The Sörensen uptake capacity of coated wound dressings.

FIG. 2: Degradation curves of dextran solutions and dextran aldehyde solutions.

FIG. 3a: A side view of a two-layer wound dressing composed of a collagen layer and of a polysaccharide layer composed of dextran and dextran aldehyde.

FIG. 3b: A two-layer wound dressing composed of a collagen layer and a polysaccharide layer composed of dextran and dextran aldehyde.

FIG. 4: A side view of a three-layer wound dressing composed of two polysaccharide layers, each consisting of dextran and dextran aldehyde, and of a collagen layer located in between.

FIG. 5: A SE micrograph of a dextran aldehyde layer of a wound dressing.

FIG. 6: A SE micrograph of a wound dressing in cross section.

FIG. 7: A SE micrograph of a collagen layer of a wound dressing.

### DETAILED DESCRIPTION

We provide a wound dressing having at least one biocompatible support material and at least one polysaccharide, in particular for use as hemostatic means (hemostyptic) and/or for uniting severed regions of body tissue, the wound dressing having a layered structure of at least one layer of the support material (support layer) and at least one layer of the polysaccharide (polysaccharide layer).

Our wound dressings make it possible to improve the absorbability of the polysaccharide and in particular thus to increase the biocompatibility of the wound dressing to a particular extent. This is achieved in particular by a quite considerable reduction in the amount of polysaccharide in the wound dressing. The polysaccharide layer can be kept so thin that it is ho longer self-supporting. Despite this, the polysaccharide is present in concentrated and in particular undiluted form oh the surface of the wound dressing, so that it is able to exert its full hemostatic and in particular adhesive effect. The absorbability of the polysaccharide is particularly preferably specifically influenced, in particular accelerated, via its degree of oxidation. The improved absorbability of the polysaccharide, in particular the accelerated absorption of the

polysaccharide, distinctly increases me biocompatibility of the wound dressing.

A “layered structure” means a structure of layers, i.e., of regions which are extended two-dimensionally and have a substantially uniform (homogeneous) structure.

A “support material” means a material which confers on the wound dressing, especially in a moist environment, an adequate stability, in particular mechanical stability, with retention of flexibility.

The term “biocompatibility” means the property of a material, in particular of a substance, of not causing on contact with body regions, in particular with body tissues, any undesired side effects, in particular significant traumatizations, for example in the form of necroses.

The term “absorbability” means the rate of absorption of a substance, i.e., the rate of its degradation inside and outside the human and/or animal body. The term “absorbability” is intended in particular to mean the uptake of a substance into the bloodstream and the subsequent excretion of the substance by the organs of excretion.

In one construction, the wound dressing may have a support layer and polysaccharide layer with a common interface, with, preferably, opposite surfaces of the support layer and polysaccharide layer being in contact.

In another construction, the wound dressing has a multilayer structure. It is thus particularly preferred for the wound dressing to have a three-layer structure. The wound dressing may thus have a three-layer structure, in which case the support layer is preferably located between two polysaccharide layers. It is possible in this way particularly advantageously for separate regions of body tissue, preferably organs, to be united, in particular bonded.

In one construction, the wound dressing has a two-layer structure. The wound dressing preferably comprises a support layer and a polysaccharide layer.

The support layer and polysaccharide layer of the wound dressing are preferably connected to one another. The connection between the support layer and polysaccharide layer particularly preferably exists through a common contact area. The connection between the support layer and polysaccharide layer of the wound dressing is essentially based on non-covalent linkages, in particular on Van-der-Waals forces, hydrogen bonds and electrostatic interactions. The cohesion of the layered structure of the wound dressing is particularly advantageously ensured in this way. In particular, detachment or slippage of individual layers of the wound dressing is avoided.

The wound dressing can be in particular in the form of a bonded Web. The wound dressing preferably has a layered structure with a fibrous web support layer and a fibrous web polysaccharide layer. A wound dressing of this type can be produced in particular by needling and rolling the fibers. The support layer of the wound dressing may additionally have a porous and/or fibrous structure, and the polysaccharide layer may have a structure differing therefrom, in particular a lamellar structure.

In a further construction, the support layer and polysaccharide layer of the Wound dressing are present as layers configured substantially separately from one another. “Configured substantially separate from one another” means that slight overlaps between the support layer and polysaccharide layer may be present in the region between the support layer and polysaccharide layer, in particular over a common interface. The support layer and polysaccharide layer of the wound dressing are preferably present as layers configured completely separate from one another but with a common interface. An unwanted, in particular functional, impairment of the individual layers of the wound dressing is thus particularly advantageously precluded.

The support layer of the wound dressing is preferably free of the polysaccharide of the polysaccharide layer. The stabilizing properties of the support layer are thus in particular not impaired. This particularly advantageously increases the stability, especially the mechanical stability, of the wound dressing.

In a further preferred construction, the polysaccharide layer of the wound dressing is free of the support material of the support material layer. The polysaccharide is thus present preferably undiluted, i.e., concentrated, in the polysaccharide layer. The hemostatic and, in particular, adhesive properties of the polysaccharide are thus utilized in a particularly advantageous manner.

In a particularly preferred construction of the wound dressing, the polysaccharide layer is configured as flat dressing for a wound. This is particularly advantageous because the polysaccharide of the polysaccharide layer preferably has better hemostatic properties and in particular better adhesive properties by comparison with the support material.

In a further and particularly preferred construction, the support material of the wound dressing is a polymeric support material. The support material is particularly preferably absorbable. This is particularly preferred because the biocompatibility of the wound dressing is increased overall in this manner.

In a further construction, the support material is formed of at least one protein, in particular of at least one recombinant protein. The support material of the wound dressing is preferably formed of at least one protein of animal origin. The support material of the wound dressing is particularly preferably collagen. Collagen is a naturally occurring, fibrous and very durable structural protein, which is why collagen is particularly preferred as support material for the wound, dressing. In a further construction, the support material of the wound dressing is partly denatured collagen, preferably gelatin. The collagen may be collagen of type I, II, III and/or IV, preferably of type I.

The protein of the wound dressing is preferably of bovine, porcine and/or equine origin. The protein of the wound dressing is particularly preferably of equine origin. By comparison with proteins from other animal sources, proteins of equine origin cause a small risk of transmission of pathogens to a recipient organism.

The support material of the wound dressing may be in particular a polysaccharide, for example cellulose, or a polysaccharide derivative. The polysaccharide is advantageously oxidized. The polysaccharide is preferably oxidized cellulose, in particular carboxymethylcellulose (CMC).

In a further particularly preferred construction, the support material of the wound dressing is a synthetic polymer. The polymer may for example be polyvinyl alcohol (PVA) or polyethylene glycol (PEG). The polymer of the wound dressing is preferably a co- or terpolymer, preferably based on lactide, glycolide, ε-caprolactone, trimethylene carbonate and/or p-dioxanone.

In a particularly preferred construction, the polysaccharide of the wound dressing is at least one polysaccharide from the group of dextran, starch, amylose, amylopectin, chitosan, cellulose, chondroitin sulfate, hyaluronic acid, alginate and derivatives thereof. The polysaccharide of the wound dressing is preferably at least one polysaccharide from the group of dextran, starch and chitosan. Chitosan is preferred because of its wound-healing and, in particular, antimicrobial properties. In a particularly preferred construction, the polysaccharide of the wound dressing is dextran. Dextran is particularly preferred as material for the polysaccharide layer of the wound dressing because of its good absorbability and, in particular, because of its high biocompatibility. A further advantage derives from the good adhesive properties of dextran vis-à-vis regions of body tissue.

In a further and particularly preferred construction, the polysaccharide layer of the wound dressing has groups reactive with body tissues, in particular carboxyl, aldehyde and/or alcohol groups, preferably aldehyde groups. The presence of groups reactive with body tissues in the polysaccharide layer contribute in an advantageous manner to an improvement in the adhesive properties of the polysaccharide layer and thus of the wound dressing. The groups reactive with body tissues of the polysaccharide layer are preferably present only on the surface thereof. The groups reactive with body tissues are thus particularly advantageously present in concentrated and, in particular, undiluted form. This has the advantageous effect of improving the hemostatic properties and, in particular, the adhesive properties of the wound dressing.

In a preferred construction, the polysaccharide of the polysaccharide layer is oxidized. The polysaccharide is preferably a polysaccharide having carboxyl and/or aldehyde groups, preferably a polysaccharide having exclusively aldehyde groups (exclusively polyaldehydic polysaccharide). The polysaccharide may be for example oxidized by treatment with periodic acid or a salt of periodic acid, in particular sodium periodate. The monosaccharide units present in the polysaccharide preferably each have two aldehyde groups.

In a further preferred construction, the oxidized polysaccharide of the wound dressing has a low degree of oxidation. This is particularly advantageous because the absorbability depends crucially on the degree of oxidation of the oxidized polysaccharide. A lower degree of oxidation of the oxidized polysaccharide means that the absorption of the polysaccharide proceeds faster. It is particularly preferred for the polysaccharide layer to consist of an oxidized polysaccharide having a low degree of oxidation. A low degree of oxidation of the oxidized polysaccharide thus contributes in a particularly advantageous manner to increasing the biocompatibility of the wound dressing.

The degree of oxidation of the oxidized polysaccharide is intended to mean the proportion of monosaccharide units which are in oxidized form in the oxidized polysaccharide.

In a particularly preferred construction of the wound dressing, about 10 to about 90%, in particular about 10 to about 50%, preferably about 10 to about 30%, of the monosaccharide units in the polysaccharide are in oxidized form. It is particularly preferred for about 25% of the monosaccharides in the polysaccharide derivative to be in oxidized form.

In a preferred construction, the polysaccharide layer of the wound dressing includes in addition to the polysaccharide a polysaccharide derivative, preferably a derivative of the polysaccharide. The polysaccharide layer of the wound dressing preferably consists, in addition to the polysaccharide, of the polysaccharide derivative, preferably of a derivative of the polysaccharide. The polysaccharide derivative is preferably a polysaccharide having carboxyl groups. The polysaccharide derivative is particularly preferably a polysaccharide having aldehyde groups. The polysaccharide derivative is preferably a polysaccharide having exclusively aldehyde groups (exclusively polyaldehydic polysaccharide). This has the effect in particular of reducing the amount of polysaccharide derivative, in particular of slowly absorbable polysaccharide derivative, in the polysaccharide layer. The rate of absorption of the polysaccharide layer is particularly effectively increased in this way.

Polysaccharide and polysaccharide derivative are preferably present in the polysaccharide layer of the wound dressing as a mixture. The mixture is preferably in the form of a type of solid dispersion, with the polysaccharide preferably forming the solid dispersion medium in which the polysaccharide derivative is in particular dispersed. It is particularly advantageously possible to reduce the amount of polysaccharide derivative in the polysaccharide layer and thus overall in the wound dressing through the dispersed distribution of the polysaccharide derivative in the polysaccharide layer.

In another particularly preferred construction, the polysaccharide layer itself has a layered structure composed of so-called “sublayers.” The sublayers preferably comprise a sublayer of a polysaccharide and a sublayer of a polysaccharide derivative. The sublayers of the polysaccharide layer are preferably present as sublayers configured substantially separately from one another. It is particularly preferred for the sublayer of the polysaccharide derivative to be present on the surface of the polysaccharide layer of the wound dressing. This is particularly preferred because of the particular hemostatic properties and, in particular, because of the adhesive properties of the polysaccharide derivative, especially in the case of a polysaccharide having aldehyde groups. Concerning further features of the sublayers of the polysaccharide layer, reference is made to the description hitherto concerning the layered structure of the wound dressing.

It is further preferred for the polysaccharide and the polysaccharide derivative in the polysaccharide layer to be present in different ratios of amounts relative to each other. It is advantageous to choose the ratio of amounts so that the amount of polysaccharide is larger than the amount of polysaccharide derivative. The absorption of the polysaccharide layer is thus particularly advantageously increased. The ratio of amounts of polysaccharide to polysaccharide derivative in the polysaccharide layer is preferably in a range from about 50% by weight of polysaccharide to about 50% by weight of polysaccharide derivative up to about 90% by weight of polysaccharide to about 10% by weight of polysaccharide derivative, based on the total weight of the polysaccharide layer of the wound dressing.

In a particularly preferred construction, the polysaccharide layer of the wound dressing includes dextran and/or dextran aldehyde. The polysaccharide layer of the wound dressing preferably includes dextran and dextran aldehyde. It is particularly preferred for the polysaccharide layer of the wound dressing to consist of dextran and/or dextran aldehyde, preferably of dextran and dextran aldehyde. As already mentioned, dextran is particularly preferred as material for the polysaccharide layer of the wound dressing in particular because of its high biocompatibility. Dextran aldehyde is preferred in particular because of its hemostatic and wound-bonding properties.

In a further construction, the wound dressing comprises active substances. The active substances may be present in the support layer and/or in the polysaccharide layer. The active substances are preferably antimicrobial active substances, in particular polyhexamethylene-biguanide (PHMB), thymol, chlorhexidine salts, furanone derivatives, triclosan, chitosan and/or antibiotics. PHMB is advantageously present as salt, preferably as hydrochloride. Examples of possible chlorhexidine salts are chlorhexidine diacetate, chlorhexidine digluconate and/or chlorhexidine dihydrochloride. Preferred as antibiotics are in particular gentamycin and/or rifampicin. The antimicrobial active substances may further be biocompatible metals, preferably silver, or biocompatible metal compounds, in particular silver acetate. The metals are preferably in the form of nanoparticles. The metals may further be present in a colloidal state.

In a further particularly preferred construction, the active substances are antiinflammatory active substances, in particular allatoin, saponin, riboflavin, flavonoids, tocopherol, betasitosterol, Soledum-cineol, dexpanthenol and/or bromalain. The flavonoids may be in particular nobiletin, rutin and/or hesperedine.

In a further construction, the wound dressing is lyophilized. The lyophilization makes it possible particularly advantageously to shape the wound dressing permanently. The wound dressing preferably has a weight per unit area of from about 55 to about 180 g/m², in particular from about 100 to about 170 g/m², preferably from about 120 to about 140 g/m².

It is particularly preferred for the support layer of the wound dressing to have a weight per unit area of from about 50 to about 140 g/m², preferably from about 80 to about 120 g/m². It is further particularly preferred for the polysaccharide layer of the wound dressing to have a weight per unit area of from about 5 to about 40 g/m², preferably from about 5 to about 20 g/m².

In a further preferred construction, the support material of the wound dressing has a larger layer thickness than the polysaccharide layer. The greater thickness of the support layer by comparison with the polysaccharide layer of the wound dressing has the effect in particular of improving the stability, especially the mechanical stability, of the wound dressing. The support material of the wound dressing preferably has a layer thickness of from about 0.5 to about 8 mm, in particular from about 1 to about 5 mm, preferably from about 2 to about 4 mm.

The polysaccharide layer of the wound dressing particularly advantageously has a layer thickness of from about 0.07 to about 5 mm, in particular from about 0.07 to about 1 mm, preferably from about 0.07 to about 0.5 mm. The distinctly smaller layer thickness of the polysaccharide layer, by comparison with the layer thickness of the support material, contributes in particular to improving the biocompatibility of the wound dressing.

In a further particularly preferred construction, the wound dressing is absorbable. The wound dressing is preferably absorbable within about 6 to about 12 weeks. This is particularly advantageous because the wound area to be treated is not exposed over a prolonged period to the wound dressing materials in this way. This reduces in particular the risk of traumatization of the body regions to be treated and increases in a particularly effective manner overall the biocompatibility of the wound dressing.

It is further preferred for the wound dressing to be absorbent. The absorbency of the wound dressing particularly advantageously has the effect of taking up tissue fluid in the region of the wound area to be treated and, in this way, promotes successful treatment of the wound. The absorbent properties of the wound dressing additionally have the effect of taking up wound fluid (exudate). In this way, accumulation of wound fluid in the region of the wound area is prevented and, in particular, the risk of infection is distinctly reduced.

It is particularly preferred for the wound dressing to have ah adequate wet stability. The wound dressing is preferably swellable in aqueous liquids, preferably in physiological liquids. The wound dressing can particularly advantageously take up liquids in an amount corresponding to a multiple of the wound dressing's own weight. An adequate stability of the wound dressing on contact with liquids, in particular with body fluids, is ensured in this way.

In a further construction, the wound dressing is flexible. In this way, the wound dressing can particularly advantageously also be applied to wound surfaces which are not flat, in particular to convex wound surfaces.

In a preferred construction, the wound dressing has at least one, preferably one, colored layer. The polysaccharide layer of the wound dressing is preferably colored. The layer of the wound dressing is colored in particular with biocompatible colorants. The layer of the wound dressing can be colored for example with so-called “D&C colorants” (Drug & Cosmetic Colorants). The layer coloring of the wound dressing serves in particular to improve the identifiability of the layers and in particular to simplify differentiating the support layer from the polysaccharide layer which is preferably provided as contact surface for a wound.

The wound dressing is moreover preferably in sterilized and/or aseptically packaged form.

We further provide processes for producing a wound dressing having at least one biocompatible support material and at least one polysaccharide, in particular a wound dressing comprising the steps:
providing at least one dispersion of the support material in a liquid dispersion medium,
cooling and solidifying (freezing) the dispersion to form at least one solid layer of the support material (support layer),
applying the polysaccharide to the solid support layer,
cooling and solidifying (freezing) the applied polysaccharide to form at least one solid layer of the polysaccharide on the support layer,
removing the dispersion medium to unite the layers together.

A “dispersion” is intended also expressly to mean a solution.

In a preferred embodiment of the process, the dispersion medium is an aqueous liquid. Water or mixtures of water and water-soluble organic solvents, in particular alcohols, preferably isopropanol, is particularly preferably used as dispersion medium. A mixture of water and isopropanol is preferably used as dispersion medium. A dispersion medium mixture of water and isopropanol in a ratio of about 70% by weight of water to about 30% by weight of isopropanol, in particular of about 95% by weight of water to about 5% by weight of isopropanol up to about 80% by weight of water to about 20% by weight of isopropanol, preferably of about 87.5% by weight of water to about 12.5% by weight of isopropanol, based on the total weight of the dispersion medium mixture, is preferably used. The use of isopropanol as ingredient of a dispersion medium mixture is particularly preferred for producing a homogeneous dispersion of the support material. It is thus possible particularly advantageously to produce a homogeneous dispersion of a protein of animal origin, preferably of collagen, by using a mixture of water and isopropanol. In other cases, the use of one dispersion medium, preferably of water, may be sufficient to produce a homogeneous dispersion, in particular of the support material. It is thus possible in particular to produce a homogeneous dispersion of gelatin on use of water as dispersion medium.

The dispersion of the support material is preferably transferred, in particular to remove the dispersion medium, into a shaping environment, preferably into a lyophilization dish.

In a further aspect of the process, a dispersion of the support material, preferably a protein of animal origin, is provided with a concentration of the support material of from about 0.5 to about 5% by weight, in particular from about 1 to about 4% by weight, preferably from abpit 1.5 to about 2.5% by weight, based on the total weight of the dispersion. The dispersion of the support material is preferably produced as suspension in the process.

In a further aspect of the process, the polysaccharide is applied in solid form, in particular as powder, to the solid support layer.

In a particularly preferred aspect of the process, the polysaccharide is provided as a dispersion in a liquid dispersion medium for application to the solid support layer. Concerning the dispersion medium, reference is made to the previous description, with water being preferred as dispersion medium. The polysaccharide dispersion is preferably provided with a concentration of the polysaccharide of from about 1 to about 5% by weight, in particular from about 1.5 to about 3.5% by weight, preferably of about 2% by weight, based on the volume of the polysaccharide dispersion. The polysaccharide dispersion is preferably provided in the form of a polysaccharide solution.

In a further preferred aspect of the process, the polysaccharide dispersion is sprayed, onto the support layer. It is possible in this way particularly advantageously to apply a particularly thin polysaccharide layer, preferably a layer of a polysaccharide having exclusively aldehyde groups, to the surface of the support layer. This is particularly preferred in relation to the biocompatible properties of the wound dressing produced by the process. In another aspect of the process, the polysaccharide dispersion is poured onto the support layer.

In a further particularly preferred aspect of the process, the cooling and solidifying, i.e., the freezing, of the dispersion of the support material and, in particular, of the polysaccharide, preferably of the polysaccharide dispersion, is carried out in a temperature range between about −10° C. and about −50° C., in particular between about −20° C. and about −40° C., preferably at about −30° C.

It is particularly advantageous in the process to remove the dispersion medium by lyophilization. In a case which is particularly preferred, where the polysaccharide is provided as a dispersion in a liquid dispersion medium, the dispersion medium of the support layer and the polysaccharide dispersion are removed, whereupon the support layer and polysaccharide layer are united together. A temperature gradient is preferably used for the lyophilization. The lyophilization is preferably carried out in a temperature range between about −30° C. and about +30° C.

We also provide a wound dressing which is produced or can be produced by one of the processes. We further provide for the use of the wound dressing as a hemostatic means (hemostyptic) and/or as adhesive. The wound dressing is preferably used to unite severed regions of body tissue. The wound dressing can advantageously be configured and employed in the manner of a tissue connector. The wound dressing is preferably used for treating human and/or animal wounds, preferably internal wounds, particularly preferably internal bleeding wounds. The wounds to be treated may be, in particular, wounds following a body tissue resection, lesion, biopsy and/or rupture. The wound dressing is particularly preferably employed for treating wounds of body organs, especially of the liver. The wound dressing is particularly advantageously used for treating leaks, in particular lung leaks.

The wound dressing is particularly advantageously distinguished by a distinctly increased biocompatibility. This is achieved in particular by the wound dressing making do With distinctly smaller amounts of polysaccharides, in particular of slowly absorbable polysaccharide derivatives, by comparison with the wound dressings known in the prior art. This improves in a particularly advantageous manner the absorbability of the polysaccharide layer. The absorbability of the polysaccharides is further improved particularly preferably by a low degree of oxidation of the polysaccharides. The risk of traumatizations in the region of the wound area after application of the wound dressing is thus distinctly reduced or entirely avoided. The polysaccharides, in particular polysaccharide derivatives, are further provided in concentrated form through the layered structure of the wound dressing, whereby they are able optimally to display their hemostatic and in particular wound-sealing properties. This advantageously contributes to a successful and, in particular, low-risk wound management.

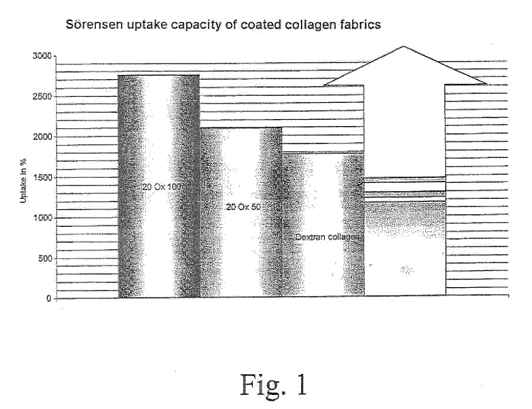

FIG. 1 shows the swellability (wet stability) of wound dressings produced in various ways with a collagen layer as support layer is depicted. The left-hand bar represents the swellability of a wound dressing whose polysaccharide layer consists exclusively of dextran aldehyde with a degree of oxidation of 100% (all the monosaccharide units present in the dextran aldehyde are oxidized). The second bar from the left represents the swellability of a wound dressing whose polysaccharide layer consists exclusively of dextran aldehyde with a degree of oxidation of 50% (half of the monosaccharide units present in the dextran aldehyde are oxidized). The second bar from the right represents the swellability of a wound dressing whose polysaccharide layer consists exclusively of dextran. The right-hand arrow represents the swellability of a wound dressing produced according to Example 3. The arrow is intended to indicate that determination of the swellability or wet stability of the wound dressing produced according to Example 3 is impossible. The wound dressings whose swellability are depicted in FIG. 1 were produced in accordance with Example 4. It is thus unambiguously evident from FIG. 1 that our wound dressings have, in contrast to the wound dressing produced according to Example 3, a wet stability which can be determined and is at the same time extremely hight.

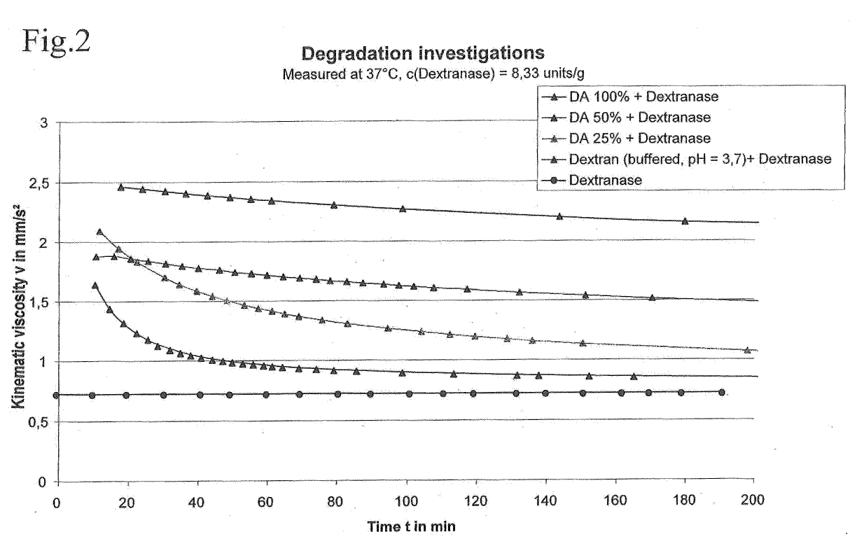

FIG. 2 shows the degradation of dextran and dextran aldehyde (DA) with different degrees of oxidation (DA 25%, DA 50% and DA 100%) on incubation with the enzyme dextranase is represented graphically. The kinematic viscosity v [mm²] is plotted on the ordinate of the graph, the time t [min] is plotted on the abscissa. 3% strength (w/v) aqueous polysaccharide solutions (extra-pure water) were produced and stored at 37° C. The solutions were mixed with dextranase (4.17 units per g of substrate), the decrease in viscosity [η=f (MW)] was then examined by capillary viscometry at different times. It can clearly be inferred from the graph that the rate of absorption of dextran aldehyde increases as the degree of oxidation falls.

The graph represents the following, from top to bottom: the topmost curve (black triangles) describes the degradation behavior of dextran aldehyde with a degree of oxidation of 100%. The second curve from top (mid-gray triangles) describes the degradation behavior of dextran aldehyde with a degree of oxidation of 50%. The third curve from top (pale gray traingles) describes the degradation behavior of dextran aldehyde with a degree of oxidation of 25%. The second curve from the bottom (dark gray triangles) describes the degradation behavior of pure dextran (buffered, pH=3.7). The lowest curve (black circles) corresponds to the enzyme dextranase.

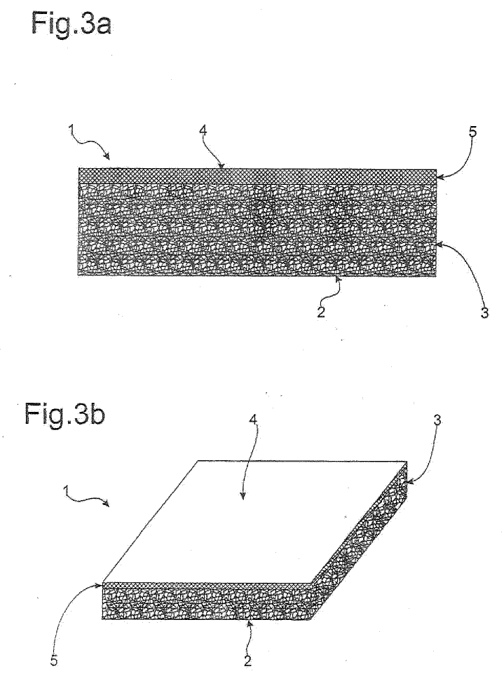

FIG. 3a shows a two-layer wound dressing 1 whose support layer 2 consists of collagen and has a layer thickness 3 of about 3 mm is depicted. The collagen layer 3 is coated on one side on its surface with a polysaccharide layer 4 composed of dextran and dextran aldehyde, with the collagen layer 2 and the polysaccharide layer 4 being present as layers completely separate from one another. The polysaccharide layer 4 of the wound dressing 1 of the invention has a layer thickness 5 of about 0.2 mm.

FIG. 3b shows a two-layer wound dressing 1 whose support layer 2 consists of collagen and has a layer thickness 3 of about 3 mm is depicted. The collagenous support layer 2 is coated oh one side on its surface with a polysaccharide layer 4 composed of dextran and dextran aldehyde. The layer thickness 5 of the polysaccharide layer 4 of the wound dressing 1 is about 0.2 mm. The surface formed by the polysaccharide layer 4 of the wound dressing 1 is particularly advantageously suitable as contact surface for a wound area.

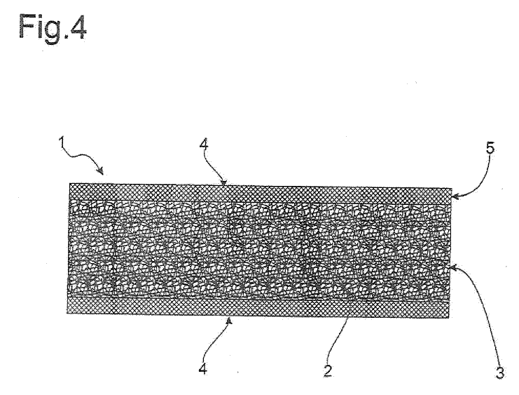

FIG. 4 shows a three-layer wound dressing 1 whose support layer 2 consists of collagen and has a layer thickness 3 of about 4 mm is depicted. The collagenous support layer 3 is coated on two sides on its surface with a polysaccharide layer 4 composed of dextran and dextran aldehyde, with the collagenous support layer 2 and the polysaccharide layers 4 being present as layers completely separate from one another. The polysaccharide layer 4 of the wound dressing 1 has a layer thickness 5 of about 0.2 mm. The wound dressing 1 of the invention is particularly suitable for uniting mutually opposite and in particular severed regions of body tissue.

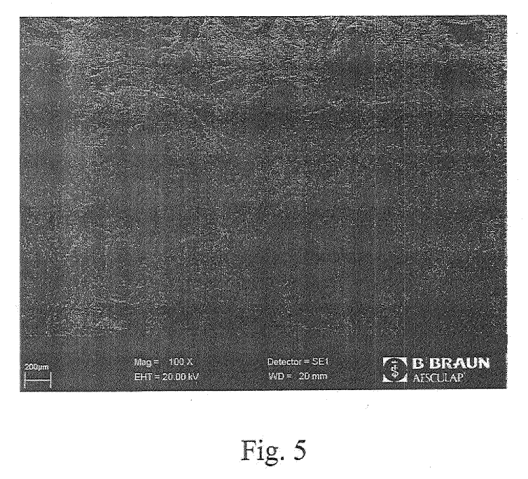

FIG. 5 shows a SE micrograph of a dextran aldehyde layer of a wound dressing. The SE micrograph reveals a leaflike structure of the dextran aldehyde layer.

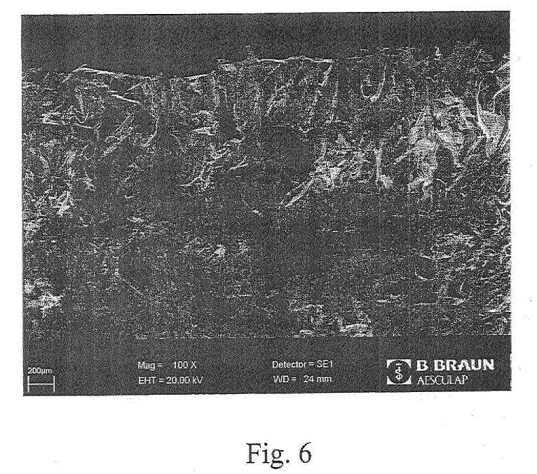

FIG. 6 shows a SE micrograph of a wound dressing composed of a collagen layer and dextran aldehyde layer is shown in cross section. The SE micrograph clearly reveals a layered structure of the wound dressing composed of a collagen layer and a dextran aldehyde layer, with the layers being configured separate from one another but present over a common interface. The upper layer of the depicted wound dressing constitutes the dextran aldehyde layer. The lower layer of the depicted wound dressing constitutes the collagen layer.

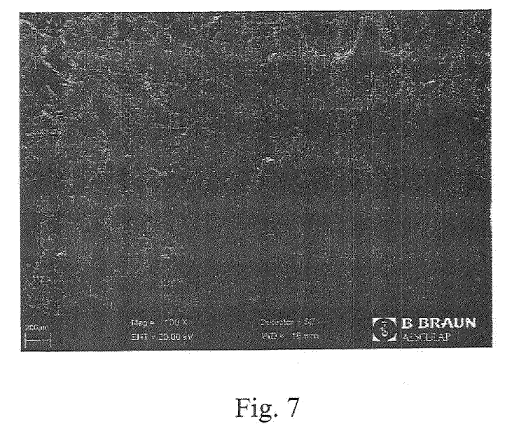

FIG. 7 shows a SE micrograph of a collagenous support layer of a wound dressing. The SE micrograph reveals a fibrous structure of the collagenous support layer.

### EXAMPLE 1

### Spraying of Collagen Fabrics

Lyostypt® fabrics (5×5 cm²) were sprayed by means of a spray bottle with in each case 2 ml of a 2% strength (w/v) dextran aldehyde solution (degree of oxidation 50%). Part of the fabrics was then dried in a drying oven at about 37° C., while the other part of the fabrics was frozen and lyophilized. The dried fabrics shrank irrespective of the drying process and were overall very brittle.

### EXAMPLE 2

### Dipping of Collagen Fabrics

Lyostypt® fabrics (5×5 cm²) were dipped in a 2% strength (w/v) dextran aldehyde solution (degree of oxidation 50%) for 10, 20 and 30 seconds. Part of the fabrics was then dried in a drying oven at about 37° C., while the other part of the fabrics was frozen and lyophilized. The dried fabrics shrank irrespective of the drying process and were overall very brittle.

### EXAMPLE 3

### Production of a Wound Dressing by Mixing a Collagen Suspension and a Polysaccharide Solution

66 g of collagen were swollen in 850 ml of extra-pure water (MilliQ water, from Millipore, Germany). 500 ml of an aqueous 2% strength (w/v) dextran aldehyde solution (degree of oxidation 50%) were added to 685 ml of water. The collagen swollen in extra-pure water was then added to the aqueous dextran aldehyde solution from water and suspended therein for 20 minutes. Thereafter 65 g portions of the suspension were poured into lyophilization dishes with a base area of about 165 cm², frozen and then lyophilized. A wound dressing lacking a layered structure and lacking wet stability was obtained.

### EXAMPLE 4

### Production of a Two-Layer Wound Dressing

66 g of collagen were swollen in 850 ml of extra-pure water (MilliQ water, from Millipore, Germany). The swollen collagen was then suspended in 1150 ml of extra-pure water for 2 minutes. Thereafter, 65 g portions of the suspension were poured into lyophilization dishes with a base area of about 165 cm² and deep-frozen at −30° C. for 60 minutes. Then 25 g of a 2% strength (w/v) aqueous polysaccharide solution (with mixing variations as shown in Table 1) Were homogeneously poured onto the frozen collagen suspension. Immediately after the polysaccharide solution had been distributed on the frozen collagen suspension, the contents of the dish were again frozen at −30° C. and then lyophilized. It was possible in this way to obtain a wound dressing with a layered structure, with the protein layer and polysaccharide layer being present substantially separate from one another and having a common area of contact.

TABLE 1
Possible mixing ratios of dextran aldehyde and dextran and possible degree of oxidation
of dextran aldehyde for producing a dextran aldehyde/dextran solution according
to Example 4, and abbreviations for the amount and the degree of oxidation of dextran
aldehyde (a Ox. b., where a indicates the amount of dextran aldehyde [100 mg]
and b the degree of oxidation of dextran aldehyde [%])

| Amount of dextran aldehyde/dextran in | Degree of | Degree of | Degree of |
|---|---|---|---|
| 100 ml of solution (in g) | oxidation 100% | oxidation 50% | oxidation 25% |
| 2 g/0 g | 20 Ox. 100 | 20 Ox. 50 | 20 Ox. 25 |
| 1.5 g/0.5 g | 15 Ox. 100 | 15 Ox. 50 | 15 Ox. 25 |
| 1 g/1 g | 10 Ox. 100 | 10 Ox. 50 | 10 Ox. 25 |
| 0.5 g/1.5 g | 5 Ox. 100 | 5 Ox. 50 | 5 Ox. 25 |
| 0.2 g/1.8 g | 2 Ox. 100 | 2 Ox. 50 | 2 Ox. 25 |

| | |
|---|---|
| 0 g/2 g | Dextran/collagen |

### EXAMPLE 5

### Production of a Two-Layer Wound Dressing

66 g of collagen were swollen in 850 ml of extra-pure Water (MilliQ water, from Millipore, Germany). The swollen collagen was then suspended in 1150 ml of extra-pure water for 20 minutes. Thereafter, 65 g portions of the suspension were poured into lyophilization dishes with a base area of about 165 cm² and deep-frozen at −30° C. for 60 minutes. Then 10 g of a 2% strength (w/v) aqueous polysaccharide solution were homogeneously distributed by means of a spray bottle on the frozen collagen suspension. Immediately after the polysaccharide solution had been distributed on the frozen collagen suspension, the contents of the dishes were again frozen at −30° C. and then lyophilized. A two-layer wound dressing which had a particularly thin and homogeneous layer of the polysaccharide on the collagen layer was obtained.

### EXAMPLE 6

### Swellability and Uptake Capacity in a Sörensen Buffer Solution

The wound dressings obtained from Examples 4 and 5 were cut into pieces 2×2 cm² in size. Their dry weight W_{dry} was then determined. The wound dressings were then dipped by means of an aspiration needle in about 20 ml of a Sörensen buffer solution with a pH of 7.4 for 30 seconds. The wet weight W_{wet} was then determined, and the degree of swelling X was calculated with the aid of the following equation:

TABLE 2
Comparison of degree of swelling in a Sörensen buffer
solution of different wound dressings produced according to
Examples 3 and 4
$X = \frac{{W}_{wet} - {W}_{dry}}{{W}_{dry}} \times 100.$

| | | | Dextran/ | Wound dressing of |
|---|---|---|---|---|
| | 20 Ox 100 | 20 Ox 50 | collagen | Example 3 |
| Degree of | 2750% | 2100% | 1770% | cannot be determined |
| swelling (X) | | | | because disintegrated |
| | | | | in solution |

### EXAMPLE 7

### Swellability and Uptake Capacity in a Hemoglobin Solution

The wound dressings were cut into pieces 2×2 cm² in size and their dry weight W_{dry} was determined. The wound dressings were then placed in about 20 ml of a 15% strength (w/v) hemoglobin solution for 4 hours. The wet weight W_{wet} was then determined and the degree of swelling X was calculated with the aid of the following equation:

$$X = \frac{{W}_{wet} - {W}_{dry}}{{W}_{dry}} ⋆ 100.$$

Subsequently, the wound dressings were dried to constant weight W_{HG} in a drying oven at 37° C., and the hemoglobin uptake capacity was calculated by the following equation:

TABLE 3
Comparison of degree of swelling in hemoglobin solution and
hemoglobin uptake capacity of different wound dressings
produced according to Examples 3 and 4
${X}_{HG} = \frac{{W}_{HG} - {W}_{dry}}{{W}_{dry}} \times 100.$

| | | | Wound dressing of |
|---|---|---|---|
| | 20 Ox 100 | 20 Ox 50 | Example 3 |
| Degree of swelling (X) | 2836% | 2931% | cannot be determined |
| Hemoglobin uptake | 345% | 359% | because disintegrated |
| capacity (X_{HG}) | | | in solution |

### EXAMPLE 8

### Partial Resection of Liver in Rabbits

Anesthetized rabbits were subjected to an abdominal incision and exposure of the animals' livers. To induce parenchymal bleeding, a standardized partial resection was performed on one of the lobes of the liver. The size of the wound dressings was adapted to the wound area so that adequate covering of the edges of the wound was ensured. After application of the wound dressings, a light manual pressure was applied to the wound for 30 seconds. Each of the wound dressings employed (wound dressing with the abbreviation 20 Ox 100, wound dressing with the abbreviation 10 Ox 25, wound dressing with the abbreviation 2 Ox 25, dextran/collagen wound dressing, cf. Table 1) was tested on 5 animals. The hemorrhages were stopped within 30 to 50 seconds. The wound dressings showed no significant differences in terms of their efficiency. In parallel thereto, a conventional surgical pad was likewise pressed with light manual pressure onto the wound and adequate hemostasis could not be achieved after about 180 seconds. The animals were sacrificed after 28 days. Whereas no residues of the three wound dressings—dextran/collagen wound dressing, wound dressing with the abbreviation 10 Ox 25 and wound dressing with the abbreviation 2 Ox 25 (cf. Table 1)—were to be seen on the wound, residues of the wound dressings with the abbreviation 20 Ox 100 (cf. Table 1) could still be found under the microscope.

### EXAMPLE 9

### Stopping Liver Hemorrhages in Pigs

After the animals had been anesthetized, an abdominal incision was performed and the lobes of the liver were exposed. To induce parenchymal bleeding, a 2×2 cm² and 0.5 cm thick piece of liver was removed. The size of the wound dressings was adapted to the wound area so that adequate covering of the edges of the wound was ensured. After application of the wound dressings, a light manual pressure was applied to the wound for 60 seconds. Each wound dressing (wound dressing with the abbreviation 20 Ox 100, wound dressing with the abbreviation 10 Ox 25, wound dressing with the abbreviation 2 Ox 25, dextran/collagen wound dressing, cf. Table 1) was tested in two animals on two different lobes of the liver in each case. The hemorrhages were stopped within 50 to 120 seconds. The wound dressings showed ho significant differences in terms of their efficiency. In parallel with this, a conventional surgical pad was likewise pressed with light manual pressure onto the wound. Adequate hemostasis could not be achieved after 300 seconds. The animals were sacrificed 6 weeks after the operation. Whereas once again no wound dressing residues were evident by microscopy oh the wound in the animals treated with the dextran/collagen wound dressing, wound dressing with the abbreviation 10 Ox 25 and the wound dressing with the abbreviation 2 Ox 25 (cf. Table 1), residues of the wound dressings with the abbreviation 20 Ox 100 (cf. Table 1) were still detectable on the livers of the animals treated therewith.

1-36. (canceled)

37. A wound dressing comprising at least one biocompatible support material and at least one polysaccharide as a hemostatic means (hemostyptic) and/or that unites severed regions of body tissue, and having a layered structure of at least one layer of the support material and at least one layer of the polysaccharide.

38. The wound dressing as claimed in claim 37, wherein the support and polysaccharide layers have a common interface, with opposite surfaces of the support and polysaccharide layers being in contact.

39. The wound dressing as claimed in claim 37, having a multilayer structure.

40. The wound dressing as claimed in claim 37, wherein the wound dressing has a two-layer structure.

41. The wound dressing as claimed in claim 39, consisting of the support layer and the polysaccharide layer.

42. The wound dressing as claimed in claim 37, wherein the support layer and polysaccharide layer are present as layers configures separate from one another.

43. The wound dressing as claimed in claim 37, wherein the polysaccharide layer is configured as flat dressing for a wound.

44. The wound dressing as claimed in claim 37, wherein the support material is formed of at least one protein of animal origin.

45. The wound dressing as claimed in claim 37, wherein the support material is formed of collagen.

46. The wound dressing as claimed in claim 37, wherein the support material is a synthetic polymer.

47. The wound dressing as claimed in claim 46, wherein the synthetic polymer is a co- or terpolymer based on lactide, glycolide, ε-caprolactone, trimethylene carbonate and/or p-dioxanone.

48. The wound dressing as claimed in claim 37, wherein the polysaccharide is at least one selected from the group consisting of dextran, starch, amylase, amylopectin, chitosan, cellulose, chondroitin sulfate, hyaluronic acid, alginate and derivatives thereof.

49. The wound dressing as claimed in claim 37, wherein the polysaccharide layer has groups reactive with body tissues.

50. The wound dressing as claimed in claim 37, wherein the polysaccharide layer has aldehyde groups.

51. The wound dressing as claimed in claim 37, wherein the polysaccharide is an oxidized polysaccharide.

52. The wound dressing as claimed in claim 37, wherein the polysaccharide is a polysaccharide having exclusively aldehyde groups.

53. The wound dressing as claimed in claim 51, wherein about 10 to about 90% of the monosaccharide units present in the polysaccharide are in oxidized form.

54. The wound dressing as claimed in claim 37, wherein the polysaccharide layer includes dextran and/or dextran aldehyde.

55. The wound dressing as claimed in claim 37, wherein the wound dressing comprises active substances.

56. The wound dressing as claimed in claim 37, wherein the wound dressing is lyophilized.

57. The wound dressing as claimed in claim 37, wherein the wound dressing has a weight per unit area of from about 100 to about 140 g/m².

58. The wound dressing as claimed in claim 37, wherein the support layer has a weight per unit area of from about 50 to about 140 g/m².

59. The wound dressing as claimed in claim 37, wherein the polysaccharide layer has a weight per unit area of from about 5 to about 40 g/m².

60. The wound dressing as claimed in claim 37, wherein the support material has a larger layer thickness than the polysaccharide layer.

61. The wound dressing as claimed in claim 37, wherein the support material has a layer thickness of from about 0.5 to about 8 mm.

62. The wound dressing as claimed in claim 37, wherein the polysaccharide layer has a layer thickness of from about 0.07 to about 5 mm.

63. The wound dressing as claimed in claim 37, wherein the wound dressing is absorbable.

64. The wound dressing as claimed in claim 37, wherein the wound dressing is swellable in aqueous liquids.

65. A process for producing a wound dressing comprising at least one biocompatible support material and at least one polysaccharide comprising:
providing at least one dispersion of the support material in a liquid dispersion medium,
cooling and solidifying the dispersion to form at least one solid layer of the supporting material,
cooling and solidifying the applied polysaccharide to form at least one solid layer of the polysaccharide on the support layer, and removing the dispersion medium to unite the layers together.

66. The process as claimed in claim 65, wherein the liquid dispersion has a concentration of the support material of from about 0.5 to about 5% by weight, based on the total weight of the dispersion.

67. The process as claimed in claim 65, wherein the polysaccharide is provided as a dispersion in the liquid dispersion medium for application to the solid support layer.

68. The process as claimed in claims 67, wherein the polysaccharide dispersion is provided with a concentration of the polysaccharide of from about 1 to about 5% by weight.

69. The process as claimed in claim 67, wherein the polysaccharide dispersion is sprayed onto the support layer.

70. The process as claimed in claim 67, wherein the polysaccharide dispersion is poured onto the support layer.

71. The process as claimed in claim 65, wherein cooling and solidifying the dispersion of the support material and the polysaccharide is carried out in a temperature range between about −10° C. and about −50° C.

72. The process as claimed in claim 65, wherein the dispersion medium is removed by lyophilization.

73. the process as claimed in claim 72, wherein lyophilization is carried out in a temperature range between about −30° C. and +30° C.

74. A wound dressing produced by the process as claimed in claim 65.

75. the process as claimed in claim 65, wherein the wound dressing is a hemostatic means (hemostyptic) and/or an adhesive.
